(19) United States
(12) Patent Application Publication
(10) Pub. No.: US 2019/0127675 A1
(43) Pub. Date: May 2, 2019

(54) MICROFLUIDIC CHIP MODULES, SYSTEMS, AND METHODS FOR IMPROVING AIR QUALITY

(71) Applicant: Artveoli, Inc., San Jose, CA (US)

(72) Inventors: Alina Adams, Santa Clara, CA (US); Anastasia Neddersen, San Jose, CA (US)

(21) Appl. No.: 15/963,380

(22) Filed: Apr. 26, 2018

Related U.S. Application Data
(63) Continuation of application No. PCT/US16/58713, filed on Oct. 25, 2016.
(60) Provisional application No. 62/246,196, filed on Oct. 26, 2015.
(60) Provisional application No. 62/333,644, filed on May 9, 2016.

(51) Int. Cl. C12M 3/06; C12M 1/00; C12M 1/34

(52) U.S. Cl. CPC C12M 23/16; C12M 29/04; C12M 21/02; C12M 41/34; C12M 23/58

(57) ABSTRACT

Methods and systems are provided for removing a component from air. A microfluidic chip comprising a fluid flow path in fluid communication with at least one surface comprising at least one phototrophic organism is provided. Additionally, air is brought in contact with the at least one surface comprising said at least one phototrophic organism. Further, said component is removed from said air with said at least one phototrophic organism.

### CROSS-REFERENCE

This application is continuation of PCT Application PCT/US2016/058713, filed on Oct. 25, 2016 which claims the benefit of U.S. Provisional Application No. 62/246,196, filed Oct. 26, 2015, and U.S. Provisional Application No. 62/333,644, filed May 9, 2016, which are both incorporated herein by reference.

### BACKGROUND

Indoor air quality is worsening around the world, especially in the industrial cities and metropolitan areas. Many people spend over 90% of their time indoors. Noxious oxides, including carbon dioxide, are major indoor air contaminants. Elevated indoor carbon dioxide levels degrade air quality and have been tied to headaches, drowsiness, nausea, fatigue, increased respiration rate, trouble concentrating, dizziness, restlessness, and irritation of the eyes, nose, throat, and lungs.

### SUMMARY

Provided herein are devices, systems, and methods, for improving air quality using microfluidic chips that contain phototrophic organisms.

In one aspect, a method of removing a component from air is provided. The method comprises providing a microfluidic chip comprising a fluid flow path in fluid communication with at least one surface comprising at least one phototrophic organism. The method also comprises bringing air in contact with the at least one surface comprising said at least one phototrophic organism. Additionally, the method comprises removing said component from said air with said at least one phototrophic organism.

In another aspect, a microbial air purification system is provided. The microbial air purification system comprises an air exchange surface area of less than 1 m² and phototrophic organisms in contact with the air exchange surface area, wherein the phototrophic organisms lower a concentration of a component in air in a room having a volume greater than 1000 ft³ by at least 25% when the phototrophic organisms are exposed to light over a time period of at least 10 seconds.

In a further aspect, a microbial air purification system is provided. The microbial air purification system comprises a microfluidic chip having one or more microfluidic channels, wherein the one or more microfluidic channels contain phototrophic organisms cultured in a cell culture medium, wherein in response to light, the phototrophic organisms remove a component in air that is within the cell culture medium.

In another aspect, a microbial purification system is provided. The microbial purification system comprises a microfluidic chip having one or more microfluidic channels, wherein the one or more microfluidic channels contain phototrophic organisms cultured in a cell culture medium, wherein the phototrophic organisms remove a gas contacting the phototrophic organisms in response to exposure to light.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings or figures (also “FIG.” and “FIGs.” herein), of which:

FIG. 1 provides an exemplary illustration of a system for improving air quality using phototrophic organisms to remove a component, such as a contaminant, from air, in accordance with some embodiments.

FIG. 2 provides an exemplary illustration of a system including a design panel, microfluidic modules, a control board, a processor, a user interface element, and a cartridge, in accordance with some embodiments.

FIG. 3 provides an example of a multilayer microfluidic module, in accordance with some embodiments.

FIG. 4 provides an exemplary illustration of a system, denoting multiple microfluidic chip modules, tubing for inputs and outputs, electronic wires connecting microfluidic chip modules to the control board, a control board, cartridges, a fluidic controller, a pump, a culture reservoir, a media reservoir, and a waste reservoir, in accordance with some embodiments.

FIG. 5 provides an illustration of a multi-layer microfluidic chip module, in accordance with some embodiments.

FIGS. 6A and 6B are exemplary illustrations of cultivation and flush phases, respectively, in accordance with some embodiments.

FIG. 7 illustrates exemplary cell culturing micro-channels with the inflow of water, nutrients and live cells, separation of live cells from dead cells by a piezo disk, and the outflow of dead cells, in accordance with some embodiments.

FIG. 8 provides an exemplary illustration of cell-culturing micro-channels within a microfluidic chip, in accordance with some embodiments.

FIG. 9 provides an exemplary illustration of a chip base, in accordance with some embodiments.

FIG. 10 illustrates an exemplary microfluidic chip module, in accordance with some embodiments.

FIG. 11 provides a view of another exemplary microfluidic chip module, in accordance with some embodiments.

FIG. 12 provides a side view of an exemplary illustration of a multilayer microfluidic chip module, in accordance with some embodiments.

FIG. 13 provides a side view of another exemplary illustration of a multilayer microfluidic chip module, in accordance with some embodiments.

FIG. 14 provides another configuration of a multilayer microfluidic chip module, in accordance with embodiments.

FIG. 15 provides an exemplary illustration of a microfluidic chip with micro-holes, in accordance with some embodiments.

FIG. 16 provides an exemplary illustration of a system denoting the front design panel, the microfluidic panel behind the front design panel and a side view show the cartridge and the user interface, in accordance with some embodiments.

FIG. 17 provides an exemplary illustration of the arrangement of micro-channels in a microfluidic chip, in accordance with some embodiments.

FIG. 18 provides another exemplary illustration of the arrangement of micro-channels in a microfluidic chip, in accordance with some embodiments.

### DETAILED DESCRIPTION

The present disclosure may be understood by reference to the following detailed description, taken in conjunction with the drawings as described above. For purposes of illustrative clarity, certain elements in various drawings may not be drawn to scale, may be represented schematically or conceptually, or otherwise may not correspond exactly to certain physical configurations of embodiments.

The typical ways to provide fresh air include ventilation and filtration. Both have issues and neither can economically remove carbon dioxide from the air. Ventilation mechanically forces air to exchange with the outside or circulate air within the building. Ventilation may also occur naturally by opening windows or trickle vents in small spaces. The National Institute for Occupational Safety and Health (NIOSH) found that ventilation is often inadequate and is a primary cause of poor indoor air quality. Carbon dioxide levels are especially high where people gather, such as in conference rooms, classrooms, and inadequately ventilated homes. In such cases, carbon dioxide levels frequently rise in excess of 3,000 ppm, about three times the upper limit recommended by NIOSH and the U.S. Environmental Protection Agency (EPA).

Filtration systems force air through a filter medium to remove solid particulates, including dust, pollen, mold, and bacteria from the air. Alternatively, the filter medium may consist of an absorbent or catalyst to remove or react with airborne molecular contaminants, such as volatile organic compounds (VOCs) and ozone. Currently, the specialized filters called “carbon dioxide scrubbers” that may be used to remove carbon dioxide are expensive, often using a cold solution of various amines to bind the atmospheric carbon dioxide.

Thus, there is a need to address the ongoing problem of high carbon dioxide levels and poor indoor air quality that lies outside of the existing methods of ventilation and filtration.

Described herein are devices, systems, and methods to improve air quality using microfluidic chips that contain phototrophic organisms. In particular, the present disclosure provides microfluidic chips, microfluidic chip modules, systems comprising microfluidic chips and modules, and methods of use thereof. In some embodiments, these devices, systems, and methods can be used for improving air quality. In other embodiments, these devices, systems, and methods improve air quality by decreasing the concentration of atmospheric carbon dioxide. In other embodiments, these devices, systems, and methods improve air quality by increasing the concentration of atmospheric oxygen. In some embodiments, these devices, systems, and methods improve air quality by decreasing the concentration of atmospheric carbon dioxide and increasing the concentration of atmospheric oxygen.

In one aspect, the devices described herein comprise at least one species of phototrophic organisms. In some embodiments, the phototrophic organisms are autotrophic. In further embodiments, the phototrophic organisms can comprise a plant autotroph, an algae autotroph, and/or a bacterial autotroph. In some embodiments, the phototrophic organism is selected from cyanobacteria, algae, moss, or any combination thereof. In some embodiments, the phototrophic organisms are algae. In an exemplary embodiment, the phototrophic organisms are chlorophyte. In a further exemplary embodiment, the phototrophic organisms are Spirulina and/or Chlorella. In one embodiment, the phototrophic organisms are Chlorella vulgaris.

In one aspect, the phototrophic organisms can be cultured in the microfluidic chip device or module. In some embodiments, the phototrophic organisms can be cultured using well-known, conventional cell culturing techniques. In some embodiments, the phototrophic organisms can be cultured in growth media comprising a buffer. In an embodiment, the phototrophic organism can be cultured in Bold's Basal Medium, a phosphate buffer for freshwater algae containing 250 mg/L NaNO₃, 75 mg/L MgSO₄.7H₂O,25 mg/L NaCl, 75 mg dipotassium phosphate (K₂HPO₄), 175 mg/L monopotassium phosphate (KH₂PO₄), 25 mg/L CaCl₂.2H₂O, trace minerals (e.g., ZnSO₄.7H₂O, MnCl₂.4H₂O, MoO₃, CuSO₄.5H₂O, Co(NO₃)2.6H₂O, and boric acid (H₃BO₃)), and stabilizers (e.g., ethylenediaminetetraacetic acid (EDTA), potassium hydroxide, FeSO₄.7H₂O, and/or concentrated sulfuric acid).

In one aspect, the phototrophic organisms can reduce the level of at least one component in the air. In some embodiments, the at least one component is a contaminant. In some examples, the at least one contaminant is a noxious oxide in the air. The at least one noxious oxide may include, but is not limited to, carbon oxide (COₓ), nitrogen oxide (NOₓ) and sulfur oxide (SOₓ). In some embodiments, the at least noxious oxide is carbon monoxide. In some embodiments, the at least one noxious oxide is carbon dioxide. In other embodiments, the at least one noxious oxide is nitric oxide. In some embodiments, the at least one noxious oxide is nitrogen dioxide. In some embodiments, the at least one noxious oxide is nitrous oxide. In some embodiments, the at least one noxious oxide is sulfur monoxide. In some embodiments, the at least one noxious oxide is sulfur dioxide. In another embodiment, the at least one noxious oxide comprises a combination of different noxious oxides.

In some embodiments, the phototrophic organisms can reduce the level of at least one component in the air by absorbing the at least one component from the air. In some examples, the at least one component is selected from the group consisting of carbon monoxide and carbon dioxide. In some embodiments, the phototrophic organisms can reduce the level of at least one noxious oxide in the air by absorbing the at least one noxious oxide from the air. In other embodiments, the phototrophic organisms can reduce the level of the at least one noxious oxide from the air by absorbing the at least one noxious oxide from the air and converting it to desirable products. In some embodiments, the photographic organisms convert the noxious oxide into oxygen. In some embodiments, the at least one component comprises formaldehyde, carbon monoxide, methane, radon, hydrogen sulfide, 1,1,1-Trichloroethane, benzene, chloroform, or any combination thereof.

In another aspect, the device or apparatus comprising phototrophic organisms may comprise at least one chip module. In some embodiments, the chip module comprises one or more microfluidic chips, at least one light source, a plurality of valves, and a chip base. In certain embodiments, the at least one chip module can further comprise at least one pump, one or more sensors, one or more filters, and/or one or more cell separators.

“Chip” or “microfluidic chip” refers to a chip comprising one or more microfluidic channels. The depth of a microfluidic channel can be from about 50 μm to about 2000 μm (2 mm). In some embodiments, the depth of the microfluidic channels can be from about 50 μm to about 100 μm, from about 100 μm to about 500 μm, from about 500 μm to about 1 mm, from about 1 mm to about 1.5 mm, or from about 1.5 mm and about 2 mm. The depth of a microfluidic channel can be at least 50 μm. The depth of a microfluidic channel can be about 1 mm. In some examples, the depth of a microfluidic channel is 1 mm. In some examples, the depth of a microfluidic channel is less than 0.8 mm, 0.8 mm, 0.9 mm, 1 mm, 1.1 mm, 1.2 mm, 1.3 mm, 1.4 mm, 1.5 mm, or greater than 1.5 mm.

In some examples, the length of a microfluidic channel can be from about 10 cm to 60 cm, In some embodiments, the length of the microfluidic channel is from about 10 cm to about 20 cm, from about 20 cm to about 25 cm, from about 25 cm to about 30 cm, from about 30 cm to about 35 cm, from about 35 cm to about 40 cm, from about 40 cm to about 45 cm, from about 45 cm to about 50 cm, from about 50 cm to about 55 cm, or from about 55 cm to about 60 cm. In some examples, the length of the microfluidic channel can be at least about 10 cm. In some examples, the length of the microfluidic channel can be at most about 60 cm. In some examples, the length of the microfluidic channel can be about 13 cm.

In some examples, the width of a microfluidics channel can be from about 50 μm to about 5000 μm (5 mm). In some embodiments, the width of a microfluidic channel can be from about 50 μm to about 100 μm, from about 100 μm to about 500 μm, from about 500 μm to about 1 mm, from about 1 mm to about 1.5 mm, from about 1.5 mm to about 2 mm, from about 2 mm to about 2.5 mm, from about 2.5 mm to about 3 mm, from about 3 mm to about 3.5 mm, from about 3.5 mm to about 4 mm, from about 4 mm to about 4.5 mm, or from about 4.5 mm to about 5 mm. The width of the microfluidics channel can be at least about 50 μm. The width of the microfluidics channel can be about 5 mm. The width of the microfluidics channel can be about 1 mm. In some examples, the width of a microfluidic channel is 1 mm. In some examples, the width of a microfluidic channel is less than 0.8 mm, 0.8 mm, 0.9 mm, 1 mm, 1.1 mm, 1.2 mm, 1.3 mm, 1.4 mm, 1.5 mm, or greater than 1.5 mm.

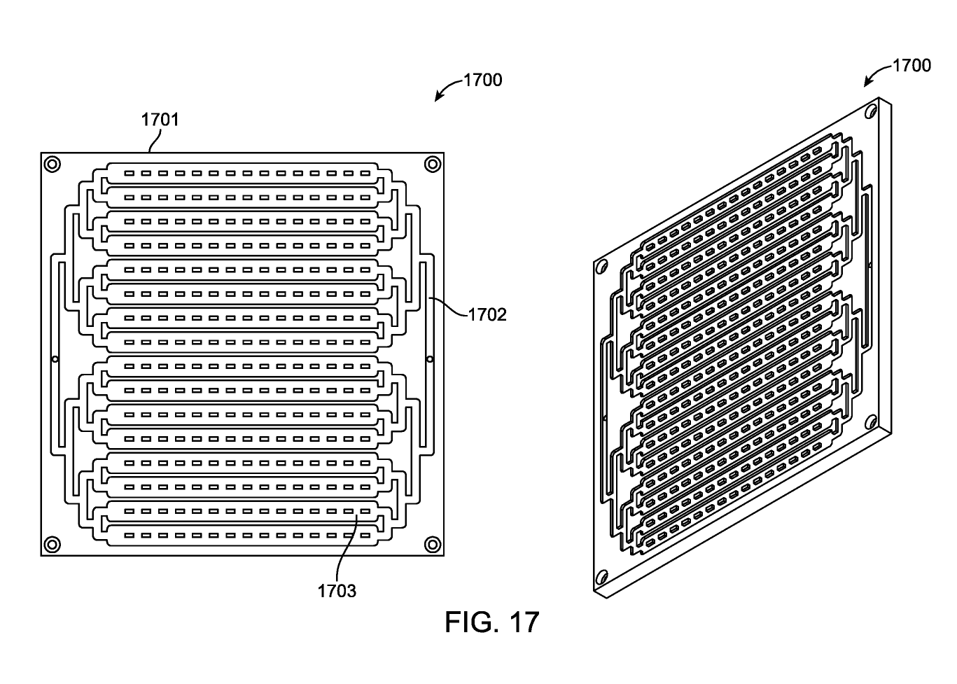

In some embodiments, the chip module can comprise a plurality of microfluidic channels. The geometry of the plurality of microfluidic channels can vary to achieve different flow rates within the microfluidic channels. In some embodiments, the plurality of microfluidic channels can be connected to each other. In some embodiments, the plurality of microfluidic channels can be elongated tubes connected to one another by a curved tube. In other embodiments, the plurality of microfluidic channels can be straight. In some embodiments, the plurality of microfluidics channels can be linear. In some embodiments, the plurality of microfluidic channels may have a more complex design. In certain embodiments, the geometry of the plurality of microfluidic channels is a multiplexed geometry (split channels) with pillars inside the channels, as shown in FIG. 17. In such embodiments, the width of the channel is about 3 mm, and separates into chambers of about 7.3 mm. In examples, a chamber having width of 7.3 mm may have a pillar with a width of about 1.3 mm and a length of about 3 mm.

The volume of a microfluidics channel depends on its dimensions and configuration. From these parameters, one of skill of the art can calculate the volume by applying the rules of geometry.

The length of a microfluidics chip comprising one or more microfluidic channels can be from about 10 cm to about 60 cm. In some embodiments, the length of a microfluidics chip can be from about 10 cm to about 15 cm, from about 15 cm to about 20 cm, from about 20 cm to about 25 cm, from about 25 cm to about 30 cm, from about 30 cm to about 35 cm, from about 35 cm to about 40 cm, from about 40 cm to about 45 cm, from about 45 cm to about 50 cm, from about 50 cm to about 55 cm, or from about 55 cm to about 60 cm. In certain embodiments, the length of the microfluidics chip can be at least about 15 cm. The length of the microfluidics chip can be at most about 60 cm. In certain embodiments, the length of the microfluidics chip is about 22 cm. The length of the microfluidics chip can be about 13 cm.

The width of a microfluidics chip comprising one or more microfluidic channels can be from about 10 cm to about 60 cm. In some embodiments, the width of a microfluidics chip can be from about 10 cm to about 15 cm, from about 15 cm to about 20 cm, from 20 cm to about 25 cm, from about 25 cm to about 30 cm, from about 30 cm to about 35 cm, from about 35 cm and about 40 cm, from about 40 cm to about 45 cm, from about 45 cm to about 50 cm, from about 50 cm to about 55 cm, or from about 55 cm to about 60 cm. The width of a microfluidics chip can be at least about 15 cm. The width of the microfluidics chip can be about 60 cm. In a certain embodiment, the width of a microfluidics chip is about 22 cm. The width of the microfluidics chip can be about 13 cm.

The microfluidics chip can be made of a transparent or optically clear material. In other embodiments, the microfluidics chip can be made of a chemically inert material. In certain embodiments, the microfluidics chip can be made of a transparent and chemically inert material. The microfluidics chip can be made of materials including, but not limited to, glass, acrylic, polycarbonate, polydimethylsiloxane (PDMS), poly(methyl methacrylate) (PMMA), polycarbonate, polystyrene, acrylic or combinations thereof.

The microfluidics chip can further comprise at least one membrane that allows for the exchange of gases between the chip and the ambient environment. In some embodiments, the membrane can be permeable to atmospheric gases including but not limited to carbon dioxide, oxygen, nitrogen, and argon. In further embodiments, the membrane can be permeable to nitrogen oxides (NOx) and sulfur oxides (SOx). The membrane can comprise gas permeable materials including but not limited to polytetrafluoroethylene (PTFE), polydimethylsiloxane (PDMS), or fluorinated ethylene propylene (FEP), or any combination thereof. In certain embodiments, the membrane can comprise AeraSeal™, which is composed of 4.5-mil hydrophobic porous film with medical-grade adhesive for sealing tissue culture plates, bio-blocks, and 96-well plates, where air and gas exchange are necessary for cell growth or bacterial cultivation. AeraSeal™ allows uniform air and carbon dioxide exchange including wells near plate edges. AeraSeal™ is non-cytotoxic, highly gas permeable, easily pierceable, sterile, and recommended for temperatures from −20° C. to +80° C.

The chip module can comprise at least one light source. In some embodiments, the light source comprises light bulbs, fluorescent lights, light-emitting diode (LED) panels, etc. In some embodiments, the light source comprises light of wavelength from about 620 nm to about 780 nm. In some embodiments, the light source comprises at least one LED panel. In other embodiments, the at least one LED panel comprises light of about 630 nanometers. In further embodiments, the LED panel can be in front of a system control box for uniform lighting. In other embodiments, the LED panel can have a reflective surface on top of each chip to reflect the light back into the chip and in so doing, using less power and conserving energy. In other embodiments, the LED panel is not be in front of a system control box, and a reflective element obstructs the light emitted from LED panel from penetrating through an artistic panel and reflects it back to the chip. In certain embodiments, another light source may be added solely to backlight an artistic panel.

The LED panel can be at most the length a microfluidics chip. The LED panel can be at most the height of the microfluidics chip. The length of an LED panel can be from about 10 cm to about 60 cm. In some embodiments, the length can be from about 10 cm to about 15 cm, from about 15 cm to about 20 cm from about 20 cm to about 25 cm, from about 25 cm to about 30 cm, from about 30 cm to about 35 cm, from about 35 cm to about 40 cm, from about 40 cm to about 45 cm, from about 45 cm to about 50 cm, from about 50 cm to about 55 cm, or from about 55 cm to about 60 cm. The length of the LED panel can be at least about 15 cm. The length of the LED panel can be at most about 60 cm. In certain embodiments, an LED panel may have a length of about 22 cm.

The LED panel can be at most the width of a microfluidics chip. The width of an LED panel can be from about 10 cm to about 60 cm. In some embodiments, the width of an LED panel is from about 10 cm to about 15 cm, from about 15 cm to about 20 cm, from about 20 cm to about 25 cm, from about 25 cm to about 30 cm, from about 30 cm to about 35 cm, from about 35 cm to about 40 cm, from about 40 cm to about 45 cm, from about 45 cm to about 50 cm, from about 50 cm to about 55 cm, or from about 55 cm to about 60 cm. The width of the LED panel can be at least about 15 cm. The width of the LED panel can be at most about 60 cm. In certain embodiments, an LED panel has a width of about 22 cm. In an instance, an LED panel is about 22 cm in length, about 22 cm in width, and at most about 5 cm in depth.

The composition of light emitted from the light source is selected based on the type(s) of phototrophic organisms being cultured in the microfluidic chip(s). Different phototrophic organisms absorb light differently at different wavelengths. To determine the adequacy of the light source to promote desired metabolic functions of the phototrophic organisms, the Photosynthetic Active Radiation (PAR) is quantified in some embodiments. In other embodiments, the photon flux density (PFD) is measured. In other embodiments, the PAR and the PFD are measured. In some embodiments, the light source is selected to maximize the removal of at least one noxious oxide from the air, using PAR and/or PFD. The PAR of the light source can range from about 5 mW/cm² to about 200 mW/cm². In some embodiments, the PAR is at least about 5 mW/cm². In other embodiments, the PAR is at most about 200 mW/cm². In certain embodiments, the PAR of the light source is about 50 mW/cm².

In another aspect, the chip module comprises at least one chip base. In some embodiments, the chip comprises one or more fluidic ports. In further embodiments, the one or more fluidic ports are connected to tubing in the chip module. The chip can comprise embedded channels, or the like. In some embodiments, the plurality of valves and the at least one pump of the chip module are integrated into the chip base to recirculate cells back to the microfluidic channels of the chip. The chip base is designed to hold the microfluidic chip in place and LED panel in a specific position. The fluidic ports of the chip base connect and seal to inlet and outlet ports of the chip and to the tubing in the chip module. In some embodiments, the chip module can connect to a system frame comprising fluidic and electronic connections to the system's counterpart components.

The chip base may further comprise one or more sensors. The chip base may comprise one or more pH sensors, cell concentration sensors, temperature sensors, carbon dioxide sensors, internal pressure sensors, tracking sensors, or any combination thereof. The sensor can be a pH sensor. The sensor can be a cell concentration sensor. The sensor can be a temperature sensor. The sensor can be a carbon dioxide sensor. The sensor can be an internal pressure sensor. The sensor can be a tracking sensor. In certain embodiments, the chip base comprises at least one carbon dioxide sensor, at least one pressure sensor, and at least one temperature sensor.

The chip base can comprise one or more filters. Each filter maintains live cells within the microfluidic chips and allows smaller cells and cell debris to flow out of the microfluidic chips. In some examples, filters can comprise cellulose acetate, nylon, glass-fiber, or any combination thereof. The pore size of the filters can range from about 0.1 micrometer to about 8 micrometer. In some embodiments, the filter is a micropillar filter. In other embodiments, the filter is a glass-fiber prefilter. In some embodiments, the chip base further comprises one or more reservoirs containing beads. The beads feed through each microfluidic chip. The beads can be fed through the microfluidic chip continuously. The beads can be fed through microfluidic chip sporadically. The beads prevent clumping and/or break up any cell clumps. In some embodiments, the microbeads are magnetic. In further embodiments, the microbeads comprise an iron oxide core and a silica shell. The size of the microbeads can range from about 5 micrometers to about 500 micrometers. In some embodiments, the size of the microbeads are from about 5 um to about 10 micrometers. In other embodiments, the size of the microbeads are determined by a size of the micro-channels.

The chip base can comprise at least one input. In some embodiments, the input is in fluid communication with one or more channels of the microfluidic chips. In further embodiments, the input comprises a water input. In other embodiments, the input comprises a media input. In another embodiment, the input comprises a cleaning solution input to reset the system. In further embodiments, the input comprises sodium hypoclorite (NaOCl) to reset the system. In certain embodiments, the chip base comprises a water input, a media input, and a cleaning solution input.

In some embodiments, the chip base comprises a water input, a media inputs, and a ratio controller in fluid communication with the water input and the media input. In some embodiments, the ratio controller can control the ratio of water to media before entry of the water and the media into the channels of the microfluidic chips. In other embodiments, the ratio controller can control concentration of nutrients in the media. In further embodiments, the ratio controller can control the ration of water to media, and the concentration of nutrients in the media. The ratio controller can be in communication with the one or more sensors. The ratio controller can perform its controlling function based on the outputs from the one or more sensors. In certain embodiments, the ratio controller can adjust the concentration of nutrients in the media based on the outputs from the one or more sensors. In some embodiments, the ratio controller can control the concentration of nutrients in the media before the media enters the channels of the microfluidic chip. In other embodiments of the chip module with two or more microfluidic chips, the ratio controller can control the concentration of nutrients in media such that the concentration is different for two or more of the microfluidic chips.

The chip base can comprise at least one output. In some embodiments, the output can comprise a live cell output. In other embodiments, the output can be dead cell output. In another embodiment, the output can be a cleaning solution output to remove cleaning solution used to reset the system. In further embodiments, the chip base can comprise at least one live cell output and at least one dead cell output.

The chip base can comprise at least one cell separator. In some embodiments, the cell separator is in fluid communication with one or more channels of one or more microfluidic chips. The cell separator separates live cells into the live cell output and separates dead cells into the dead cell output.

In some embodiments, the cell separator may comprise at least one piezoelectric disk, which acoustophoretically separates live cells and dead cells. In further embodiments, the at least one piezoelectric disk can be capable of creating a voltage differential across the microfluidics channel, such that larger cells are pulled to a first side of the microfluidics channel and smaller cells and debris move to a second side of the microfluidics channel distal to the first side. A split in the microfluidics channel defining the first side and the second side separates live cells (which would be typically larger) from smaller cells, dead cells, and debris. In other embodiments, the at least one piezoelectric disk creates a voltage differential across the microfluidics channel such that larger cells are pulled to the live cell output and smaller cells and debris move to dead cell output, wherein the larger cells reenter the plurality of microfluidics channels after the separation. The live cells after separation may reenter the microfluidic channels via the input.

In other embodiments, the cell separator may comprise microbeads. In some embodiments, the microbeads are magnetic. In further embodiments, the microbeads comprise an iron oxide core and a silica shell. The size of the microbeads can range from about 5 micrometers to about 500 micrometers. In some embodiments, the size of the microbeads are from about 5 um to about 10 micrometers.

In some embodiments, the cell separator is configured for a plurality of microfluidic channels in the microfluidic chip. In certain embodiments, the cell separator comprises at least one piezoelectric disk operatively connected to a plurality of microfluidic channels and creating a voltage differential across the plurality of microfluidic channel such that live cells are pulled into a live cell output and dead cells are pulled into a dead cell output. The plurality of microfluidic channels are disposed within a microfluidic chip, are intimately connected to allow the movement of algae, are substantially in parallel to each other, and have a thickness ranging between about 50 μm and about 2 mm.

The chip module can comprise a plurality of valves. In some embodiments, the plurality of valves may control the ratio of water to nutrients in the microfluidic channels. In some embodiments, the plurality of valves can comprise needle valves, check valves, pinch valves, pneumatic flow control valves, or any combination thereof. In certain embodiments, the plurality of valves comprises needle valves. The chip module can comprise at least one pump. Suitable pumps including diaphragm pumps, syringe pumps, peristaltic pumps and the like. In some embodiments, the chip module comprises other external systems to create fluid flow within the microfluidic chip. In further embodiments, the external system can comprise an electromagnet. In some embodiments, the chip module comprises at least one pump and an external system. In other embodiments, the chip module comprises an external system and no pump.

Also provided herein are multilayer chip modules. In one aspect, the multilayer microfluidic chip modules comprising two or more microfluidic chips, two or more light sources (e.g., LED light sources), a plurality of valves, one or more control boards, and one or more chip bases. In certain embodiments, the multilayer chip module can further comprise one or more pumps, one or more sensors, one or more filters, one or more cell separators, or any combination thereof.

In some embodiments, the multilayer chip module comprises at least two microfluidic chips. In some embodiments, the multilayer chip module comprises at least five microfluidic chips. In some embodiments, the multilayer chip module comprises at least ten microfluidic chips. In some embodiments, the multilayer chip module comprises at least twenty microfluidic chips. In certain embodiments, the multilayer chip module comprises four microfluidic chips.

Each of the one or more microfluidic chips comprises one or more microfluidic channels. The depth of a microfluidic channel can be from about 50 μm to about 2000 μm (2 mm). In some embodiments, the depth of the microfluidic channels can be from about 50 μm to about 100 μm, from about 100 μm to about 500 μm, from about 500 μm to about 1 mm, from about 1 mm to about 1.5 mm, or from about 1.5 mm and about 2 mm. The depth of a microfluidic channel can be at least 50 μm. The depth of a microfluidic channel can be about 1 mm. In some examples, the depth of a microfluidic channel is 1 mm. In some examples, the depth of a microfluidic channel is less than 0.8 mm, 0.8 mm, 0.9 mm, 1 mm, 1.1 mm, 1.2 mm, 1.3 mm, 1.4 mm, 1.5 mm, or greater than 1.5 mm.

The length of a microfluidic channel can be from about 10 cm to 60 cm, In some embodiments, the length of the microfluidic channel is from about 10 cm to about 15 cm, from about 15 cm to about 20 cm, from about 20 cm to about 25 cm, from about 25 cm to about 30 cm, from about 30 cm to about 35 cm, from about 35 cm to about 40 cm, from about 40 cm to about 45 cm, from about 45 cm to about 50 cm, from about 50 cm to about 55 cm, or from about 55 cm to about 60 cm. The length of the microfluidic channel can be at least about 15 cm. The length of the microfluidic channel can be at most about 60 cm. The length of the microfluidic channel can be about 13 cm.

The width of a microfluidics channel can be from about 50 μm to about 10 mm). In some embodiments, the width of a microfluidic channel can be from about 50 μm to about 100 μm, from about 100 μm to about 500 μm, from about 500 μm to about 1 mm, from about 1 mm to about 1.5 mm, from about 1.5 mm to about 2 mm, from about 2 mm to about 2.5 mm, from about 2.5 mm to about 3 mm, from about 3 mm to about 3.5 mm, from about 3.5 mm to about 4 mm, from about 4 mm to about 4.5 mm, or from about 4.5 mm to about 5 mm. The width of the microfluidics channel can be at least about 50 μm. In some examples, the width of the microfluidics channel can be about 1 mm. In some examples, the width of the microfluidics channel can be about 5 mm. In some examples, the width of the microfluidics channel can be about 7. 3 mm. In some examples, the width of the microfluidics channel can be about 10 mm. In some instances, the microfluidics channels may be microfluidic chambers having a width of several centimeters.

In some embodiments, the chip module can comprise a plurality of microfluidic channels. The geometry of the plurality of microfluidic channels can vary to achieve different flow rates within the microfluidic channels. In certain embodiments, the plurality of microfluidic channels can be connected to each other. In certain embodiments, the plurality of microfluidic channels can be elongated tubes connected to one another by a curved tube. In other embodiments, the plurality of microfluidic channels can be straight. In other embodiments, the plurality of microfluidics channels can be linear. In some embodiments, the plurality of microfluidic channels may have a more complex design. In certain embodiments, the geometry of the plurality of microfluidic channels is a multiplexed geometry (split channels) with pillars inside the channels, as shown in FIG. 17. In such embodiments, the width of the channel is about 3 mm, and separates into chambers of about 7.3 mm. In these examples, a pillar within the chamber may have a width of about 1.3 mm and a length of about 3 mm.

The volume of a microfluidics channel depends on its dimensions and configuration. From these parameters, one of skill of the art can calculate the volume by applying the rules of geometry.

The length of a microfluidics chip comprising one or more microfluidic channels can be from about 10 cm to about 60 cm. In some embodiments, the length of a microfluidics chip can be from about 10 cm to about 15 cm, from about 15 cm to about 20 cm, from about 20 cm to about 25 cm, from about 25 cm to about 30 cm, from about 30 cm to about 35 cm, from about 35 cm to about 40 cm, from about 40 cm to about 45 cm, from about 45 cm to about 50 cm, from about 50 cm to about 55 cm, or from about 55 cm to about 60 cm. In certain embodiments, the length of the microfluidics chip can be at least about 15 cm. The length of the microfluidics chip can be at most about 60 cm. In certain embodiments, the length of the microfluidics chip is about 22 cm.

The width of a microfluidics chip comprising one or more microfluidic channels can be from about 10 cm to about 60 cm. In some embodiments, the width of a microfluidics chip can be from about 10 cm to about 15 cm, from about 15 cm to about 20 cm from 20 cm to about 25 cm, from about 25 cm to about 30 cm, from about 30 cm to about 35 cm, from about 35 cm and about 40 cm, from about 40 cm to about 45 cm, from about 45 cm to about 50 cm, from about 50 cm to about 55 cm, or from about 55 cm to about 60 cm. The width of a microfluidics chip can be at least about 15 cm. The width of the microfluidics chip can be at most about 60 cm. In a certain embodiment, the width of a microfluidics chip is about 22 cm.

The microfluidics chip can be made of a transparent or optically clear material. In other embodiments, the microfluidics chip can be made of a chemically inert material. In certain embodiments, the microfluidics chip can be made of a transparent and chemically inert material. The microfluidics chip can comprise materials including, but not limited to, glass, acrylic, polycarbonate, polydimethylsiloxane (PDMS), poly(methyl methacrylate) (PMMA), polycarbonate, polystyrene, acrylic or combinations thereof.

In some embodiments, the microfluidic chip can comprise micro- and/or nano-holes to improve the permeability and efficiency membranes. The size of the holes can range from about 10 nm to about 100 nm. The size of the holes can be at least about 10 nm. The size of the holes can be at most about 100 nm. The size of the holes can be from about 10 nm to about 20 nm; from about 20 nm to about 30 nm; from about 30 nm to about 40 nm; from 40 nm to about 50 nm; from about 50 nm to about 60 nm; from about 60 nm to about 70 nm; from about 70 nm to about 80 nm; from about 80 nm to about 90 nm; from about 90 nm to about 100 nm.

The microfluidics chip can further comprise at least one membrane that allows for the exchange of gases between the chip and the ambient environment. In some embodiments, the membrane can be permeable to atmospheric gases including but not limited to carbon dioxide, oxygen, nitrogen, and argon. In further embodiments, the membrane can be permeable to nitrogen oxides (NOx) and sulfur oxides (SOx). The membrane can comprise gas permeable materials including but not limited to polytetrafluoroethylene (PTFE), polydimethylsiloxane (PDMS), or fluorinated ethylene propylene (FEP), or any combination thereof. In certain embodiments, the membrane can comprise AeraSeal™, which is composed of 4.5-mil hydrophobic porous film with medical-grade adhesive for sealing tissue culture plates, bio-blocks, and 96-well plates, where air and gas exchange are necessary for cell growth or bacterial cultivation. AeraSeal™ allows uniform air and carbon dioxide exchange including wells near plate edges. AeraSeal™ s non-cytotoxic, highly gas permeable, easily pierceable, sterile, and recommended for temperatures from −20° C. to +80° C.

The multilayer chip module can comprise one or more light sources. In some embodiments, the light source comprises at least one light-emitting diode (LED) panel. In further embodiments, the LED panel can be in front of a system control box for uniform lighting. In other embodiments, the LED panel can have a reflective surface on top of each chip to reflect the light back into the chip and in so doing, using less power and conserving energy. In other embodiments, the LED panel is not be in front of a system control box, and a reflective element obstructs the light emitted from LED panel from penetrating through an artistic panel and reflects it back to the chip. In certain embodiments, another light source may be added solely to backlight an artistic panel.

The LED panel can be at most the length a microfluidics chip. The LED panel can be at most the height of the microfluidics chip. The length of an LED panel can be from about 15 cm to about 60 cm. In some embodiments, the length can be from about 15 cm to about 20 cm, from about 20 cm to about 25 cm, from about 25 cm to about 30 cm, from about 30 cm to about 35 cm, from about 35 cm to about 40 cm, from about 40 cm to about 45 cm, from about 45 cm to about 50 cm, from about 50 cm to about 55 cm, or from about 55 cm to about 60 cm. The length of the LED panel can be at least about 15 cm. The length of the LED panel can be at most about 60 cm. In certain embodiments, an LED panel may have a length of about 22 cm.

The LED panel can be at most the width of a microfluidics chip. The width of an LED panel can be from about 10 cm to about 60 cm. In some embodiments, the width of an LED panel is from about 10 cm to about 15 cm, from about 15 cm to about 20 cm, from about 20 cm to about 25 cm, from about 25 cm to about 30 cm, from about 30 cm to about 35 cm, from about 35 cm to about 40 cm, from about 40 cm to about 45 cm, from about 45 cm to about 50 cm, from about 50 cm to about 55 cm, or from about 55 cm to about 60 cm. The width of the LED panel can be at least about 15 cm. The width of the LED panel can be at most about 60 cm. In certain embodiments, an LED panel has a width of about 22 cm. In an instance, an LED panel is about 22 cm in length, about 22 cm in width, and at most about 5 cm in depth.

The composition of light emitted from the light source is selected based on the type(s) of phototrophic organisms being cultured in the microfluidic chip(s). Different phototrophic organisms absorb light differently at different wavelengths. In some embodiments, the wavelength of the light used is 630 nanometers. To determine the adequacy of the light source to promote desired metabolic functions of the phototrophic organisms, the Photosynthetic Active Radiation (PAR) is quantified in some embodiments. In other embodiments, the photon flux density (PFD) is measured. In other embodiments, the PAR and the PFD are measured. In some embodiments, the light source is selected to maximize the removal of at least one noxious oxide from the air, using PAR and/or PFD. The PAR of the light source can range from about 5 mW/cm² to about 200 mW/cm². In some embodiments, the PAR is at least about 5 mW/cm². In other embodiments, the PAR is at most about 200 mW/cm². In certain embodiments, the PAR of the light source is about 50 mW/cm².

In some embodiments, the multilayer microfluidic chip module comprises one or more chip bases. In some embodiments, the multilayer microfluidic chip module comprises one or more fluidic ports. In further embodiments, the one or more fluidic ports are connected to tubing in the multilayer microfluidic chip module. The one or more microfluidic chip can comprise embedded channels, or the like. In some embodiments, the plurality of valves and the at least one pump of the multilayer microfluidic chip module are integrated into the chip base to recirculate the phototrophic organisms back to the channels of the microfluidic chip. The chip base is designed to hold the microfluidic chip in place and LED panel in a specific position. The fluidic ports of the chip base connect and seal to inlet and outlet ports of the microfluidic chip and to the tubing in the chip module. In some embodiments, the chip module can connect to a system frame comprising fluidic and electronic connections to the system's counterpart components.

The multilayer chip module may further comprise one or more sensors. The chip base can comprise one or more pH sensors, cell concentration sensors, temperature sensors, carbon dioxide sensors, internal pressure sensors, tracking sensors, or any combination thereof. The sensor can be a pH sensor. The sensor can be a cell concentration sensor. The sensor can be a temperature sensor. The sensor can be a carbon dioxide sensor. The sensor can be an internal pressure sensor. The sensor can be a tracking sensor. In certain embodiments, the chip base comprises at least one carbon dioxide sensor, at least one pressure sensor, and at least one temperature sensor.

The multilayer chip module can comprise one or more filters. Each filter maintains live cells within the microfluidic chips and allows smaller cells and cell debris to flow out of the microfluidic chips. The filters can comprise cellulose acetate, nylon, glass-fiber or any combination thereof. The pore size of the filters can range from about 0.1 micrometer to about 8 micrometer. In some examples, the pore size of the filters may be less than 0.1 micrometer, 0.1 micrometer, 0.2 micrometers, 0.3 micrometers, 0.4 micrometers, 0.5 micrometers, 0.6 micrometers, 0.7 micrometers, 0.8 micrometers, 0.9 micrometers, 1 micrometer, 1.5 micrometers, 2 micrometers, 2.5 micrometers, 3 micrometers, 3.5 micrometers, 4 micrometers, 4.5 micrometers, 5 micrometers, 5.5 micrometers, 6 micrometers, 6.5 micrometers, 7 micrometers, 7.5 micrometers, 8 micrometers, or more than 8 micrometers.

In some embodiments, the filter is a micropillar filter. In other embodiments, the filter is a glass-fiber prefilter. In some embodiments, the chip base further comprises one or more reservoirs containing beads. The beads feed through each microfluidic chip. The beads can be fed through the microfluidic chip continuously. The beads can be fed through microfluidic chip sporadically. The beads prevent clumping and/or break up any cell clumps. In some embodiments, the microbeads are magnetic. In further embodiments, the microbeads comprise an iron oxide core and a silica shell. The size of the microbeads can range from about 5 micrometers to about 500 micrometers. In some embodiments, the size of the microbeads are from about 5 um to about 10 micrometers. In other embodiments, the size of the microbeads are determined by size of the micro-channels. The multilayer chip module can comprise at least one input. In some embodiments, the input is in fluid communication with one or more channels of the microfluidic chips. In further embodiments, the input comprises a water input. In other embodiments, the input comprises a media input. In another embodiment, the input comprises a cleaning solution input to reset the system. In further embodiments, the input comprises sodium hypoclorite (NaOCl) to reset the system. In certain embodiments, the chip base comprises a water input, and a media input and a cleaning solution input. In some embodiments, the multilayer chip module comprises at least one water input, at least one media input, a cleaning solution input, and at least one ratio controller in fluid communication with the water input and the media input. In some embodiments, the ratio controller can control the ratio of water to media before entry of the water and the media into the channels of the microfluidic chips. In other embodiments, the ratio controller can control concentration of nutrients in the media. In further embodiments, the ratio controller can control the ratio of water to media, and the concentration of nutrients in the media. The ratio controller can be in communication with the one or more sensors. The ratio controller can perform its controlling function based on the outputs from the one or more sensors. In certain embodiments, the ratio controller can adjust the concentration of nutrients in the media based on the outputs from the one or more sensors. In some embodiments, the ratio controller can control the concentration of nutrients in the media before the media enters the channels of the microfluidic chip. In other embodiments of the chip module with two or more microfluidic chips, the ratio controller can control the concentration of nutrients in media such that the concentration is different for two or more of the microfluidic chips.

The multilayer chip module can comprise at least one output. In some embodiments, the output can comprise a live cell output. In other embodiments, the output can be dead cell output. In another embodiment, the output can be a cleaning solution output to remove cleaning solution used to reset the system. In further embodiments, the chip base can comprise at least one live cell output, at least one dead cell output, and/or at least one cleaning solution output. The multilayer chip module can comprise at least one cell separator. In some embodiments, the cell separator is in fluid communication with one or more channels of one or more microfluidic chips. The cell separator separates live cells into the live cell output and separates dead cells into the dead cell output.

In some embodiments, the cell separator may comprise at least one piezoelectric disk, which acoustophoretically separates live cells and dead cells. In further embodiments, the at least one piezoelectric disk can be capable of creating a voltage differential across the microfluidics channel, such that larger cells are pulled to a first side of the microfluidics channel and smaller cells and debris move to a second side of the microfluidics channel distal to the first side. A split in the microfluidics channel defining the first side and the second side separates live cells (which would be typically larger) from smaller cells, dead cells, and debris. In other embodiments, the at least one piezoelectric disk creates a voltage differential across the microfluidics channel such that larger cells are pulled to the live cell output and smaller cells and debris move to dead cell output, wherein the larger cells reenter the plurality of microfluidics channels after the separation. The live cells after separation may reenter the microfluidic channels via the input. In some embodiments, the cell separator is configured for a plurality of microfluidic channels in the microfluidic chip. In certain embodiments, the cell separator comprises at least one piezoelectric disk operatively connected to a plurality of microfluidic channels and creating a voltage differential across the plurality of microfluidic channel such that live cells are pulled into a live cell output and dead cells are pulled into a dead cell output. The plurality of microfluidic channels are disposed within a microfluidic chip, are intimately connected to allow the movement of algae, are substantially in parallel to each other, and have a thickness ranging between about 50 μm and about 2 mm. In some examples, the plurality of microfluidic channels may have a thickness of 1 mm.

In other embodiments, the cell separator may comprise microbeads. In some embodiments, the microbeads are magnetic. In further embodiments, the microbeads comprise an iron oxide core and a silica shell. The size of the microbeads can range from about 5 micrometers to about 500 micrometers. In some embodiments, the size of the microbeads are from about 5 um to about 10 micrometers.

The multilayer chip module can comprise a plurality of valves. In some embodiments, the plurality of valves controls the ratio of water to nutrients in the microfluidic channels. In some embodiments, the plurality of valves can comprise be a needle valves, check valves, pinch valves, pneumatic flow control valves, or any combination thereof. In certain embodiments, the plurality of valves comprises needle valves. In further embodiments, the plurality of valves controls the ratio of water to nutrients in the microfluidic channels. The multilayer chip module can comprise one or more pumps. Suitable pumps including diaphragm pumps, syringe pumps, peristaltic pumps and the like. In some embodiments, the multilayer chip module comprises other external systems to create fluid flow within the microfluidic chip. In further embodiments, the external system can comprise an electromagnet. In some embodiments, the multilayer chip module comprises at least one pump and at least an external system. In other embodiments, the multilayer chip module comprises at least one external system and no pump.

In some embodiments, the multilayer chip module comprises two or more microfluidic chips and two or more LED panels. The two or more microfluidic chips can be in fluid communication with each other. The two or more microfluidic chips can be vertically disposed to each other. In certain embodiments, the two or more LED panels may be disposed in alternating layers with the two or more microfluidic chips. In other further embodiments, the two or more microfluidic chips can be located between the two or more LED panels and one or more reflective surfaces. The one or more reflective surfaces can face the two or more LED panels. The one or more reflective surfaces can comprise a mirror and/or a substrate having a reflective coating. The reflective coating can be capable of reflecting all the wavelengths of light emitted from the LED panel. In other embodiments, the multilayer chip comprises two or more microfluidic chips and two LED panels, with the first LED panel at a proximal end of the two or microfluidic chips and the second LED panel at the distal end of the two or more microfluidic chips.

The multilayer chip module may further comprise one or more components for regulating the temperature of the multilayer chip module. The temperature within the module can be from 15 degrees Celsius to 40 degrees Celsius. In some embodiments, the temperature within the module is 20 degrees Celsius. The one or more components can each comprise a heat sink, a Peltier cooling plate, or a fan. In some embodiments, one or more fans can be disposed along the perimeter of the multilayer chip module. The one or more fans circulate the air and aid gas exchange for each microfluidic chip. In some embodiments, the LED panels may share heat sink and/or Peltier cooling plates, when present.

The multilayer chip module may further comprise a control board. The control board is operatively connected to electronic components present in the multilayer chip module, including the pump, fan, heat sink, Peltier cooling plate, sensors, cell separator, ratio controller, and the like.

The multilayer chip module may further comprise one or more fuses operatively connected to electronic components present in the chip module.

In another aspect, two or more multilayer microfluidic modules can be configured achieve a two-dimensional or a planar structure, or a three-dimensional structure. In some embodiments, a multilayer microfluidic chip module comprises a module frame supporting two or more microfluidic chips, at least one single-sided light-emitting diode (LED) panel, at least one double-sided LED panel, a chip base, and a plurality of fans. In further embodiments, the single-sided LED panel is disposed between the two or more microfluidic chips and the chip base, and is oriented toward the top of the two or more microfluidic chips. The at least one double-sided LED panel is disposed within the two or more microfluidic chips. The plurality of fans is located along the perimeter of the module frame. The multilayer microfluidic chip may further comprise at least one heat exchanger The at least one heat exchanger can be a heat sink and/or a Peltier cooling plate. In further embodiments, the at least one double-sided LED panel can operatively connected to the at least one heat exchanger. In other embodiments, the chip base may further comprise a board operatively connected to one or more sensors, the plurality of valves, and at least one pump.

In another embodiment, a multilayer microfluidic chip module comprises a module frame supporting two or more microfluidic chips, at least one reflective surface, at least one light-emitting diode (LED) panel, at least one chip base, and a plurality of fans. The at least one reflective surface is disposed above the two or more microfluidic chips, with the at least one reflective surface being oriented toward the chip base. The at least one LED panel is disposed between the two or more microfluidic chips and the chip base and is oriented toward the reflective surface such that the light is reflected from the at least one reflective surface toward the two or microfluidic chips. The plurality of fans is configures such that the fans are disposed along the perimeter of the module frame. The multilayer microfluidic chip module may further comprise a plurality of angled reflective surfaces disposed at proximal and distal ends of the microfluidics chips, such that light from the LED panel is reflected from the perimeter of the chip module toward the microfluidic chips.

In further embodiments, a multilayer microfluidic chip module comprises a module frame supporting two or more microfluidic chips, at least one reflective surface, a first light-emitting diode (LED) panel, a second LED panel, a chip base, and a plurality of fans. The first LED panel is disposed at a proximal end of the two or more microfluidic chips and oriented toward a distal end of the two or more microfluidic chips, whereby light from the first LED panel is allowed to reach the two or more microfluidic chips. The second LED panel is disposed at the distal end of the two or more microfluidic chips and oriented toward the proximal end of the two or more microfluidic chips, whereby light from the second LED panel is allowed to reach the two or more microfluidic chips. The plurality of fans is disposed along the perimeter of the module frame.

Described herein are systems improve air quality using phototrophic organisms. In some embodiments, the system improves air quality by absorbing carbon dioxide. In other embodiments, the system improves air quality by releasing oxygen. In further embodiments, the system improves air quality by absorbing carbon dioxide, converting the absorbed carbon dioxide to oxygen, and releasing the oxygen.

These systems can decrease levels of carbon dioxide in various enclosed environments, such as houses, sports arenas, theaters, offices, laboratories, hospitals, schools, airports, train stations, bus stations, casinos, and other heavily populated or trafficked indoor areas. These systems can also be used in vehicles such as aircraft, automobiles, submarines, or spacecraft, or as component of a portable or freestanding device, such as an air freshener, air purifier, air re-circulator, and the like. Use of these systems can improve people's symptoms related to poor air quality, such as headaches, fatigue, trouble concentrating, and the like.

In some embodiments, the system comprises at least one microfluidic chip module. In other embodiments, the system comprises at least one multilayer microfluidic chip module. In further embodiments, the system comprises at least one microfluidic chip module and at least one multilayer microfluidic chip module.

The system comprises at least one system control box. The system control box comprises at least one system pump, a plurality of system valves, a plurality of reservoirs, at least one filtration cartridge, electronic components including at least a controller board, at least one power supply, a plurality of electronic cables, a plurality of connectors, a plurality of tubing and a plurality of tubing connectors. In some embodiments, the at least one system pump and the plurality of systems valves move fluids from the reservoirs to at least one microfluidic chip module and move output from the at least one microfluidic chip module into the filtration cartridge located in the system control box.

In some embodiments, the system comprises a system control box, a plurality of reservoirs, at least one mixing valve, at least one sterilization unit, at least one concentration control and a system frame. The system control box comprises at least one system pump operatively connected to at least one pump of at least one microfluidic chip module. The plurality of reservoirs are in fluid communication with the at least one microfluidic chip module. The at least mixing valve is in fluid communication with the plurality of reservoirs and with the at least one microfluidic chip module. The at least one sterilization unit is in fluid communication between the at least one microfluidic chip module and the at least one system pump, wherein dead cells and debris are removed from the at least one microfluidic chip module. The at least one concentration control is in fluid communication with an input of the at least one microfluidic chip module. The system frame defines shape and size the system and supports the at least one microfluidic chip module, the system control box, and the concentration control.

In some embodiments, the system is modular comprising of a plurality of chip modules. For example, the system can comprise 2 chip modules, 5 chip modules, 10 chip modules, 15 chip modules, 20 chip modules, or 25 chip modules. In some embodiments, the system comprises from 2 to 5 modules, from 5 to 10 modules, from 10 to 15 modules, from 15 to 20 modules, or from 20 to 25 modules. In certain embodiments, the system comprises 16 chip modules. The number of chip modules within the system may be selected with several factors in view, including the desired rate of carbon dioxide removal, the air volume of the enclosed environment where the system is located, the time-average number of people in that enclosed environment, the ventilation for the enclosure environment, the air quality outside the enclosed environment. The system can comprises two or more chip modules configured to have a two-dimensional configuration. The system can comprise two or chip modules configured to have a three-dimensional configuration.

In some embodiments, the control box is located behind the at least one LED panel of the at least microfluidic chip modules.

The dimensions of the system frame vary in accordance with the number of microfluidic chip modules in the system. Each microfluidic chip module can connect to the system frame. In some embodiments, the system has a dimension of about 1 m by about 1 m by about 10 cm. In some embodiments, the system frame guides the fluidic tubing and wiring cables to each chip module.

The system can further comprise a user interface. In some embodiments, the user interface comprises a general purpose computer, a laptop, smartphone (e.g. with Bluetooth, cellular or Internet connectivity), a touchscreen, or the like. The user interface can have a graphic user interface (GUI). In some embodiments, the user interface can comprise a sensor control, where thresholds for system operation are programmed based on targeted sensor outputs. The system can notify a user when the needs to be reset, or when components need to be replaced. The user interface can provide graphs of noxious oxide concentration over time for the enclosed environment where the system is located. The system can also monitor and/or graph other factors including temperature, pH, oxygen concentration, and cell concentration.

The system can further comprise an artistic panel. In some embodiments, the artistic panel is a three-dimensional panel with an artistic design. The artistic panel can cover one or more systems. The artistic panel can be installed on a wall, ceiling or any other flat surface. The artistic panel can have unique or custom shapes. The artistic panel can be made of a variety of materials, including poly(methyl methacrylate) (PMMA, Plexiglas™), other plastics, or metals. An LED panel of at least one chip module can be used for backlighting an artistic panel.

The system can be self-sustaining. The system can perform at least one maintenance procedure. The at least one maintenance comprises changing nutrient and media reservoirs, changing filters, resetting the system, or any combination. The system can use cleaning fluid to clean the microfluidic chip modules. The cleaning fluid can comprise an aqueous hypochlorite solution, an alcohol, organic solvents, or any combination thereof. The cleaning fluid can be 70% bleach. The cleaning fluid can be ethanol. The cleaning fluid can be isopropanol. The cleaning fluid can be acetone. In some embodiments, the system can clean the at least one microfluidic chip module and other components of the system using UV irradiation.

The system can reset to cease improving air quality. The system can reset to stop the conversion of carbon dioxide to oxygen by the phototrophic organisms. The system can reset to empty the one or more microfluidic chip modules, to run a cleaning protocol, to inoculate the one or more microfluidic chips with new cell culture, or any combinations thereof.

Described herein are methods for improving air quality methods using the microfluidic chip modules and the systems comprising phototrophic organisms described herein. In some embodiments, the methods improve air quality by absorbing noxious oxides. In some embodiments, the methods improve air quality by releasing oxygen. In certain embodiments, the methods comprising absorbing carbon dioxide, converting the carbon dioxide to oxygen, and releasing the oxygen. The one or more microfluidic chip modules, or one or more multilayer microfluidic chip modules, are installed into the system. The channels of the microfluidic chips are inoculated with cell culture comprising phototrophic organisms. The phototrophic organisms absorb noxious oxides and/or release oxygen. The system can be flushed to avoid clogging of the channels and/or tubing.

In some embodiments, the methods comprise converting carbon dioxide to oxygen. The methods comprise contacting carbon dioxide with a microfluidic chip module comprising phototrophic organisms. In further embodiments, the phototrophic organisms are algae. The phototrophic organisms use light energy, nutrients from media and the carbon dioxide diffusing through a membrane of the microfluidic chip module to produce oxygen. The oxygen diffuses out of the microfluidic chip module into the atmosphere. Any microfluidic chip module described herein is suitable for this method, including multilayer microfluidic chip modules.

During installation, the microfluidic chip module can be connected to media and/or water input tubing, and output tubing using fluidic connectors. Tubing may be selected from a Tygon™ tubing product (chemically resistant polyolefin tubing produced by Saint-Gobain Performance Plastics). The water input splits before entering the chip within the chip module.

During inoculation the system is primed with water or buffer. The initial culture may then be introduced to the microfluidic chip module using the same input as water and/or media, where valves 1 and 2 are open and valves 3 and 4 are closed. Fresh media may be introduced from the system control unit to feed the cells into all microfluidic chips. The cell culture circulates through the microfluidic chips multiple times until the phototrophic organisms reach a desired concentration, such as that based on the setting for the cell culture sensor. At this point, filtration is turned on and some cell culture leaves the chip modules via the outlet to maintain equilibrium within the cell culture.

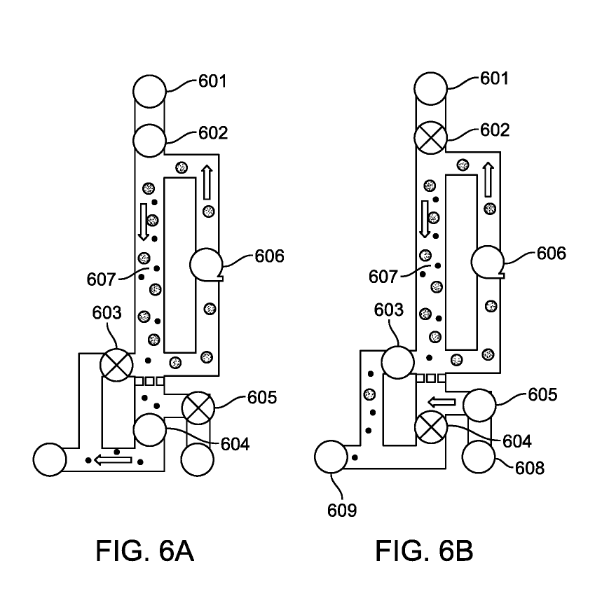

During cultivation phase (see FIG. 6A), the active photosynthesis takes place and the phototrophic organisms use the light energy, the nutrients from the media and carbon dioxide that constantly diffuses through the membrane to produce oxygen. The produced oxygen then diffuses out of the system into the surrounding air. Valves 1 and 2 are open and valves 3 and 4 are closed. The pump may be used to circulate the phototrophic organisms. Water and nutrients are continuously supplied to the phototrophic organisms via the input and to replace the water and nutrients lost via the output. Debris and dead cells may be filtered out of the microfluidic channels via the output. The live phototrophic organisms are recirculated within the plurality of microfluidic channels. This cultivation phase is maintained indefinitely, where fresh media is introduced into chip module and excess cell culture leaves chip module into the control unit filter.

To avoid clogging the channels in the microfluidic chip at the filter, a flush phase (see FIG. 6B) may be used. Valves 1 and 2 are closed and valve 3 and 4 are open. The fluid flows in the opposite direction of filters using water and/or media, thus removing the clogged phototrophic organisms from the plurality of microfluidic channels.

Each microfluidic chip module can convert from about 0.7 g and about 9 g carbon dioxide to oxygen per liter of volume of media per hour of operation (gL⁻¹h⁻¹). In some embodiments, the microfluidic chip module can convert from about 0.7 gL⁻¹h⁻¹ to about 1 gL⁻¹h⁻¹, from about 1 gL⁻¹h⁻¹t to about 2 gL⁻h⁻¹, from about 2 gL⁻¹h⁻¹ to about 3 gL⁻¹h⁻¹, from about 3 gL⁻¹h⁻¹ to about 4 gL⁻¹h⁻¹, from about 4 gL⁻¹h⁻¹ to about 5 gL⁻¹h⁻¹, from about 5 gL⁻¹h⁻¹ to about 6 gL⁻¹h⁻¹, from about 6 gL⁻¹h⁻¹ to about 7 gL⁻¹h⁻¹, from about 7 gL⁻¹h⁻¹ to about 8 gL⁻¹h⁻¹, or from about 8 gL⁻¹h⁻¹ to about 9 gL⁻¹h⁻¹.

As a matter of stoichiometry, the photosynthetic quotient of carbon dioxide to oxygen is 0.73 O₂/CO₂. To state CO₂ conversation rates in terms of O₂ output, each number is divided by 0.73. Thus, microfluidic chip module when in operation can produce about 0.5 g and about 6.5 g oxygen from carbon dioxide per liter of volume of media per hour of operation (gL⁻¹h⁻¹). In some embodiments, the microfluidic chip module can convert from about 0.5 gL⁻¹h⁻¹ to about 0.7 gL⁻¹h⁻¹, from about 0.7 gL⁻¹h⁻¹ to about 1.5 gL⁻¹h⁻¹, from about 1.5 gL⁻¹h⁻¹ to about 2.2 gL⁻¹h⁻¹, from about 2.2 gL⁻¹h⁻¹ to about 2.9 gL⁻¹h⁻, from about 2.9 gL⁻¹h⁻¹ to about 3.7 gL⁻¹h⁻¹, from about 3.7 gL⁻¹h⁻¹ to about 4.4 gL⁻¹h⁻¹, from about 4.4 gL⁻¹h⁻¹ to about 5.1 gL⁻¹h⁻¹, from about 5.1 gL⁻¹h⁻¹ to about 5.8 gL⁻¹h⁻¹, or from about 5.8 gL⁻¹h⁻¹ to about 6.5 gL⁻¹h⁻¹.

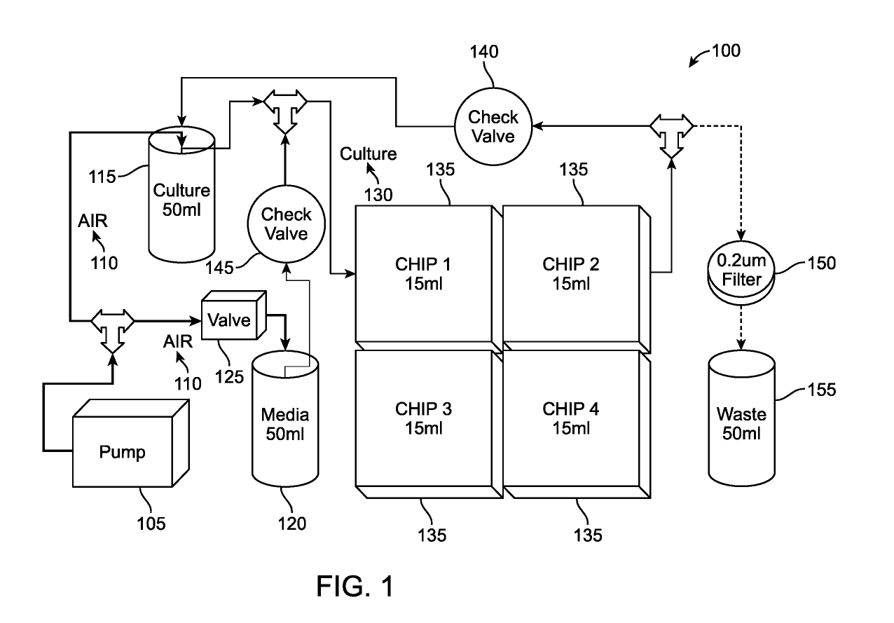

FIG. 1 provides an exemplary illustration of the system for improving air quality using phototrophic organisms to remove a component, such as a contaminant, from air, in accordance with some embodiments. In particular, FIG. 1 provides a system 100 that comprises a pump 105 that directs air 110 from the surrounding environment into two reservoirs, a culture reservoir 115 and a media reservoir 120. As seen in FIG. 1, culture reservoir 115 is a 50 mL reservoir comprising cell culture. Additionally, media reservoir 120 is a 50 mL reservoir comprising cell culture media. The cell culture further comprises phototrophic organisms. A valve 125 is disposed between the pump 105 and the media reservoir 120. Fluids, such as culture 130, comprising air from the pump 105 flow into each of four microfluidic chips 135. Each microfluidic chip 135 is capable of holding a volume of 15 mL. Additionally, a check valve 140 is disposed between the culture reservoir 115 and the microfluidic chips 135. A check valve 145 is also disposed between the media reservoir 120 and the microfluidic chips 135. Fluids also flow from each of the microfluidic chips 135. Some of the fluids flowing out of a microfluidic chip 135 are directed to the culture reservoir 115 through check valve 140. The remaining fluids flowing out of the microfluidic chips 135 are directed to a waste reservoir 155. Waste reservoir 155 is a 50 mL reservoir comprising waste. As seen in FIG. 1, fluids that flow into waste reservoir 155 pass through a 0.2 micrometer filter 150 before reaching waste reservoir 155.

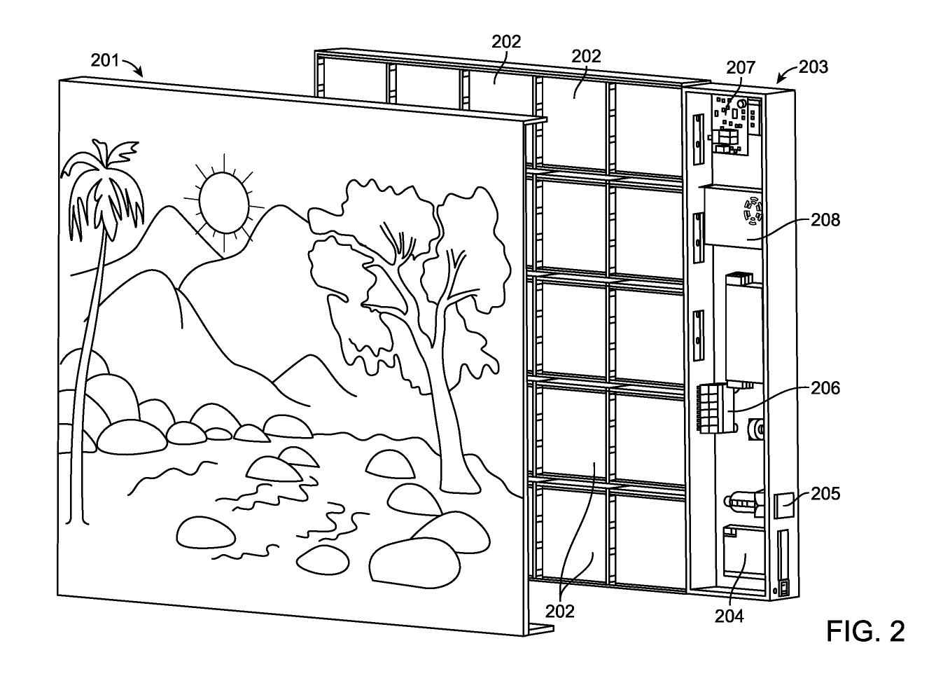

FIG. 2 provides an exemplary illustration of a system including a design panel, microfluidic modules, a control board, a processor, a user interface element, and a cartridge, in accordance with some embodiments. In particular, FIG. 2 illustrates a design panel 201. Design panel 201 may be used to cover multiple microfluidic modules 202. Located to one side of multiple microfluidic channels 202 is a support system 203. Support system 203 comprises a control board 207, a processor 208, a user interface element 205, valves and flow control 206, and a cartridge 204.

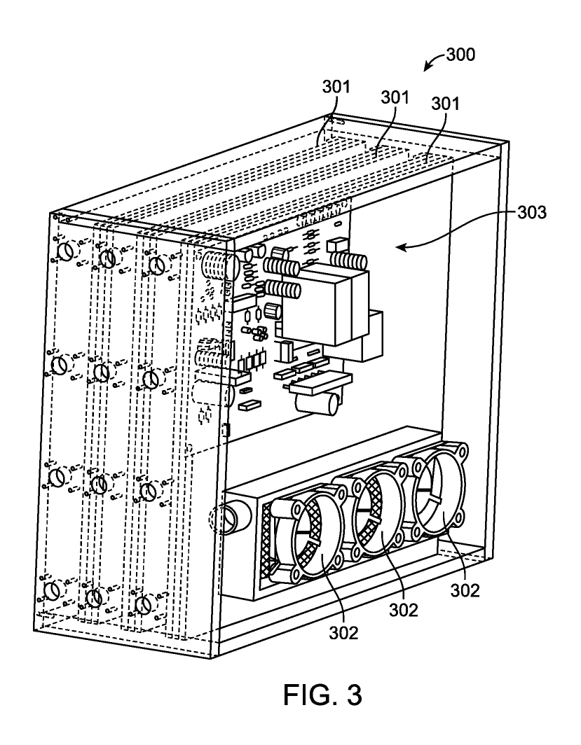

FIG. 3 provides an example of a multilayer microfluidic module 300, in accordance with some embodiments. The module 300 comprises multiple single-layer microfluidic modules 301 in vertical linearity. The control and sensor panel 303 is adjacent the microfluidic modules 301. Additionally, the fans and/or heat sinks 302 are positioned below the control and sensor panel 303.

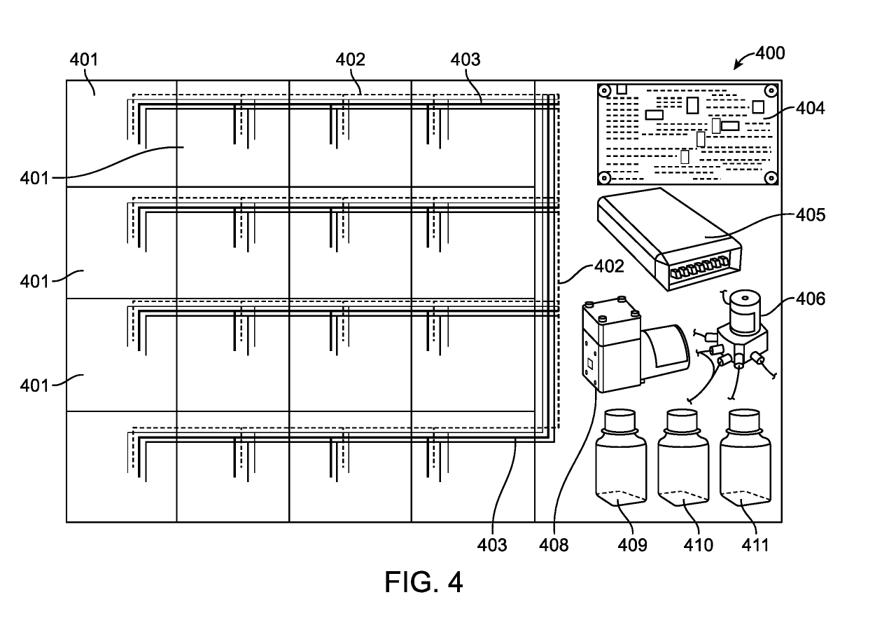

Another illustration of the system is provided in FIG. 4. In particular, FIG. 4 provides an exemplary illustration of a system 400, denoting multiple microfluidic chip modules, tubing for inputs and outputs, electronic wires connecting microfluidic chip modules to the control board, a control board, cartridges, a fluidic controller, a pump, a culture reservoir, a media reservoir, and a waste reservoir, in accordance with some embodiments. In particular, FIG. 4 illustrates sixteen microfluidic chip modules 401. The microfluidic chip modules 401 are connected to each other via tubing containing fluids 402, and electronic wiring 403 connected to the control board 404. Located to one side of the microfluidic chip modules are cartridges 405, a fluid ratio controller 406, a pump 408, and reservoirs. In particular, a culture reservoir 409 is provided for containing cell culture comprising phototrophic organisms; a media reservoir 410 is provided for containing cell culture media; and a waste reservoir 411 is provided for containing and waste.

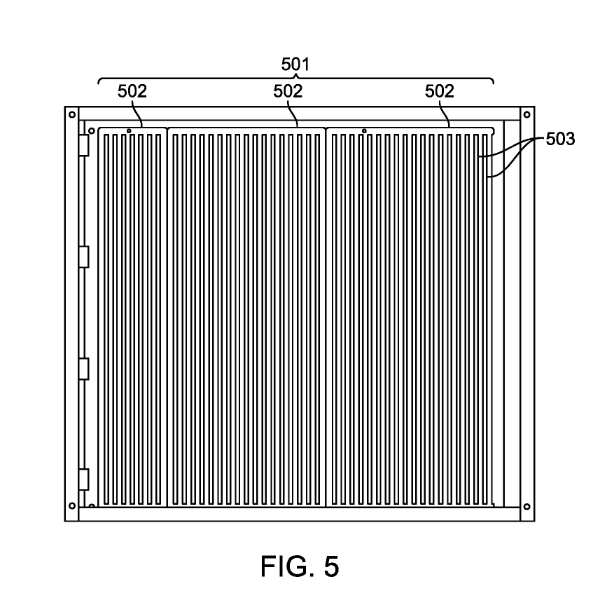

FIG. 5 provides an illustration of a multi-component microfluidic chip module 501, in accordance with some embodiments. As seen in FIG. 5, multi-component microfluidic chip module 501 has multiple single-layer microfluidic chips 502 having different geometries. Additionally, FIG. 5 illustrates multiple microfluidic channels 503 within single-layer microfluidic chips 502.

FIGS. 6A and 6B are exemplary illustrations of cultivation and flush phases, respectively, in accordance with some embodiments. During the cultivation phase as shown in FIG. 6A, cell culture media flows into the micro-channels 607 through media input 601 and open valve 602. Valves 603 and 605 are closed. A pump 606 keeps fluid circulating within the channels. A filter separates the live cells from the dead cells and cell debris. Fluid comprising dead cells and cell debris flows through the filter and open valve 604 out of the channels though the dead cells output.

During the flush phase as shown in FIG. 6B, the pump 606 is off and valves 602 and 604 are closed. Water enters the channels through the water input 608 and valve 605 to clean the channels. The water exits the channels through open valve 603 and into the dead cells output 609.

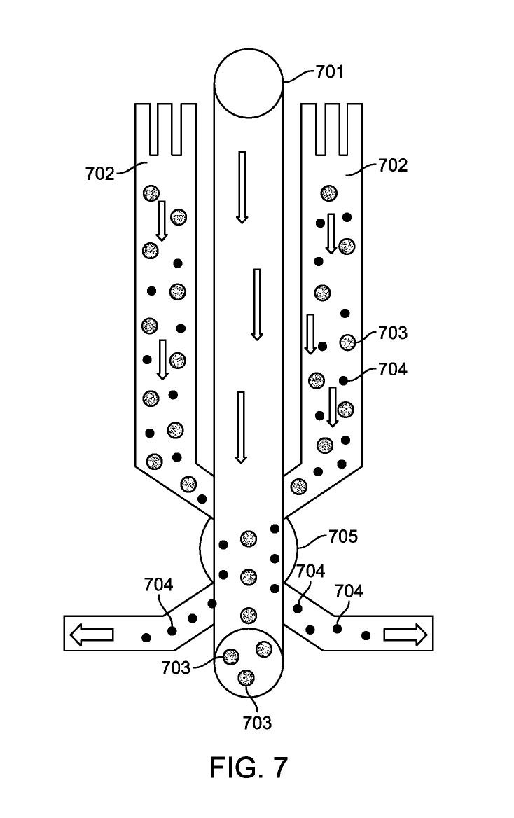

FIG. 7 provides another embodiment of fluid dynamics within the micro-channels. In particular, FIG. 7 illustrates exemplary cell culturing micro-channels with the inflow of water, nutrients and live cells, separation of live cells from dead cells by a piezo disk, and the outflow of dead cells, in accordance with some embodiments. As seen in FIG. 7, water enters the channels 702 through the water input 701. Within the cell culturing channels 702, inputs comprising water input, cell culture comprising phototrophic organisms input, and cell culture input circulate through the channels. Additionally, channels 702 contain live cells 703 and dead cells 704. A piezo disk 705 separates the fluids into live cells 703 output and dead cells 704 output as the fluids leave the micro-channels.

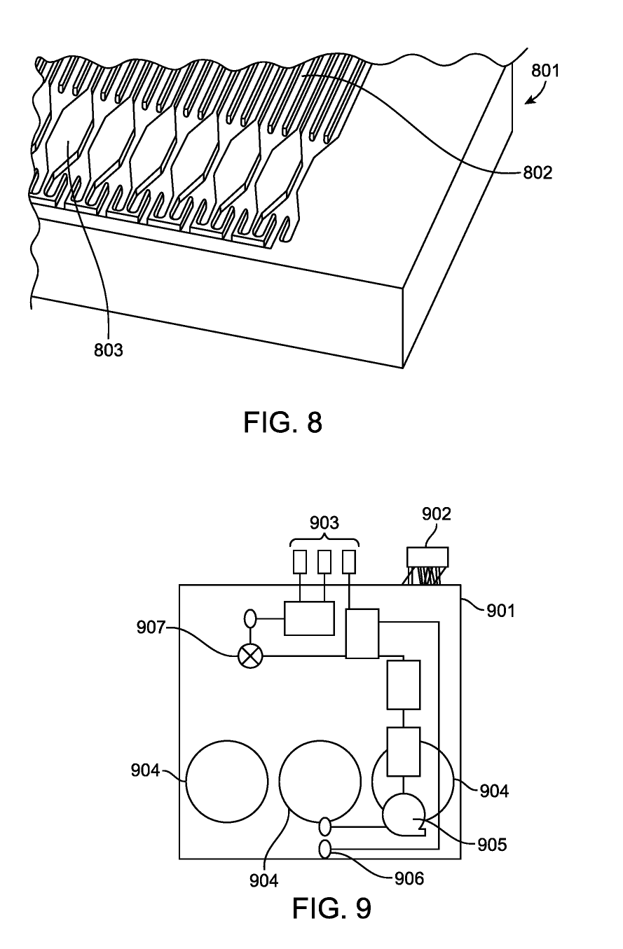

FIG. 8 provides an exemplary illustration of cell-culturing micro-channels 802 within a microfluidic chip, in accordance with some embodiments. In particular, as seen in FIG. 8, cell-culturing micro-channels 802 are configured in a linear pattern within a microfluidic chip 801. Additionally, micro-channels 802 as provided may have spacing 803 between the micro-channels 802.

FIG. 9 provides an exemplary illustration of a chip base 901, in accordance with some embodiments. Chip base 901 comprises electronic connectors 902 that connect the chip base 901 to the control board. Two inputs and one output 903 allow the flow of water and cell culture media into the chip base 901 and the flow of waste out the chip base 901. Additionally, FIG. 9 also illustrates valve 907. The chip base 901 also comprises a pressure sensor, a pH sensor and a cell density sensor which are connected in series to the pump 905. Three piezo disks 904 separate the live cells from the dead cells before they exit through the live cells output and the dead cells output 906.

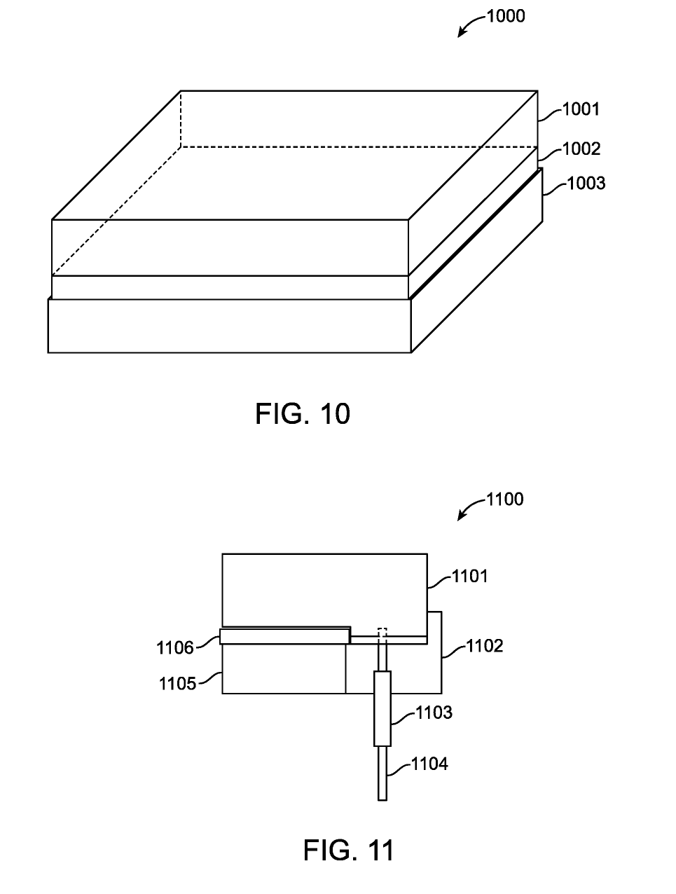

FIG. 10 illustrates an exemplary microfluidic chip module 1000, in accordance with some embodiments. In particular, FIG. 10 illustrates an exemplary microfluidic chip module 1000 comprising a clear PDMS or plastic chip 1001 at the top layer, an LED panel 1002 in the middle layer, and chip base 1003 at the lower layer.

FIG. 11 provides a view of another exemplary microfluidic chip module 1100, in accordance with embodiments. Module 1100 comprises tubing 1104 with fitting 1103 connected to a micro-channel through the chip base 1105 and the chip frame holder 1102 at the bottom. Module 1100 also comprises an LED panel 1106 between the chip base at the bottom and the clear PDMS or plastic microfluidic chip 1101 at the top.

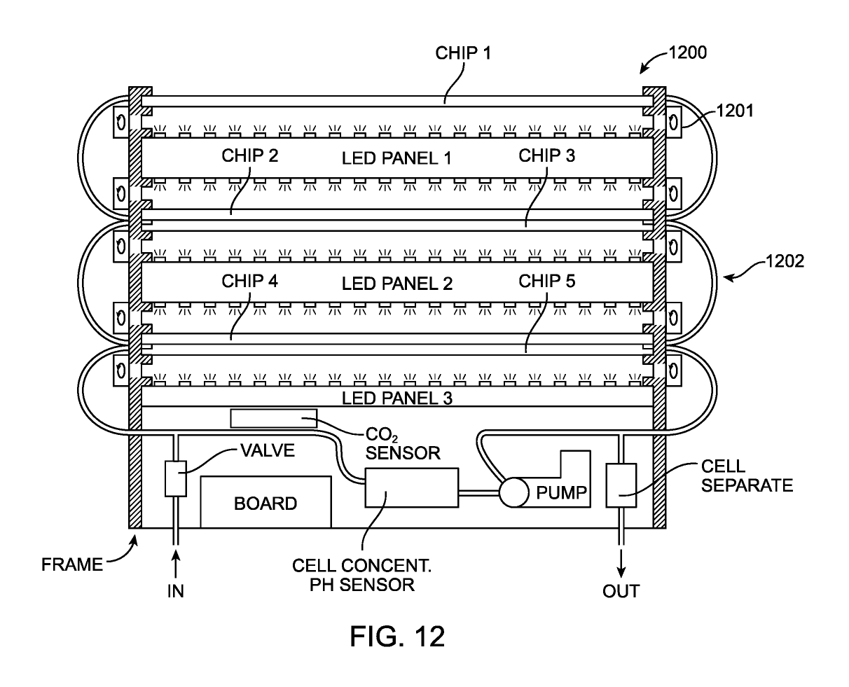

FIG. 12 provides a side view of an exemplary illustration of a multilayer microfluidic chip module 1200, in accordance with some embodiments. As seen in FIG. 12, module 1200 comprises microfluidic chip 1 at the top of module 1200. Microfluidic chips 2 and 3 are below microfluidic chip 1, with LED panel 1 disposed between them. Additionally, microfluidic chips 4 and 5 are disposed beneath microfluidic chips 2 and 3, with LED panel 2 disposed between microfluidic chips 4 and 5. LED panel 3 is below the microfluidic chips and above the chip base. The chip base comprises a control board, a carbon dioxide sensor, a pH sensor and a cell concentration sensor. The chip base also comprises a pump to assist with fluid flow in and out of the microfluidic channels. Fluid flows into the module through an input and a valve into the chip base and through multiple tubings 1202 connected to the microfluidic chip modules. The tubings are also connected to the concentration and the pH sensors and the pump. A cell separator separates the fluids into live cells output and dead cells output as the fluids exit the chip base. Fans 1201 located to the sides of the frame maintain the appropriate temperature within the module. In some examples, an appropriate temperature within the module may be 20 Celsius. In some examples, an appropriate temperature within the module may be less than 15 Celsius, 15 Celsius, 20 Celsius, 25 Celsius, 30 Celsius, 35 Celsius, 40 Celsius, or more than 40 Celsius.

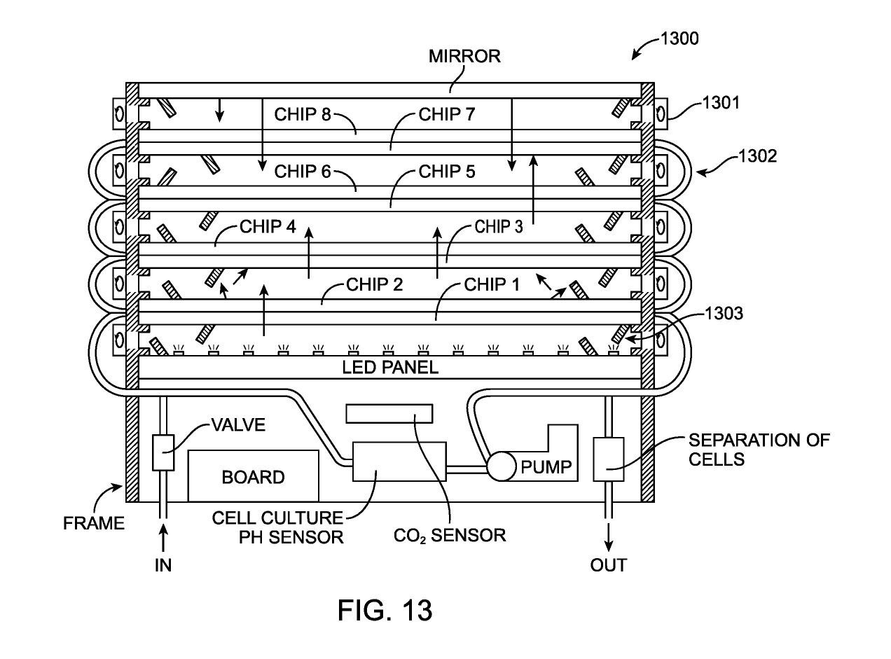

FIG. 13 provides a side view of another exemplary illustration of a multilayer microfluidic chip module 1300, in accordance with some embodiments. As seen in FIG. 13, the microfluidic chips are arranged in pairs: microfluidic chip 1 is paired with microfluidic chip 2; microfluidic chip 3 is paired with microfluidic chip 4; microfluidic chip 5 is paired with microfluidic chip 6; and microfluidic chip 7 is paired with microfluidic chip 8. Each pair is configured parallel to each other. At one end of the arrangement of microfluidic chips is an LED panel. At the other end of the arrangement is a mirror, a reflective surface to reflect light from the LED panel to the microfluidic chips. Also dispersed between the microfluidic chips are small reflective surfaces 1303 to reflect light. The chip base is located below the LED panel. It comprises a control board, a carbon dioxide sensor, a pH sensor and a cell concentration sensor. The chip base also comprises a pump to assist with fluid flow in and out of the microfluidic channels. Fluid flows into the module through an input and a valve into the chip base and through multiple tubings 1302 connected to the microfluidic chip modules. The tubings are also connected to the concentration and the pH sensors and the pump. A cell separator separates the fluids into live cells output and dead cells output as the fluids exit the chip base. Fans 1301 located to the sides of the frame maintain the appropriate temperature within the module.

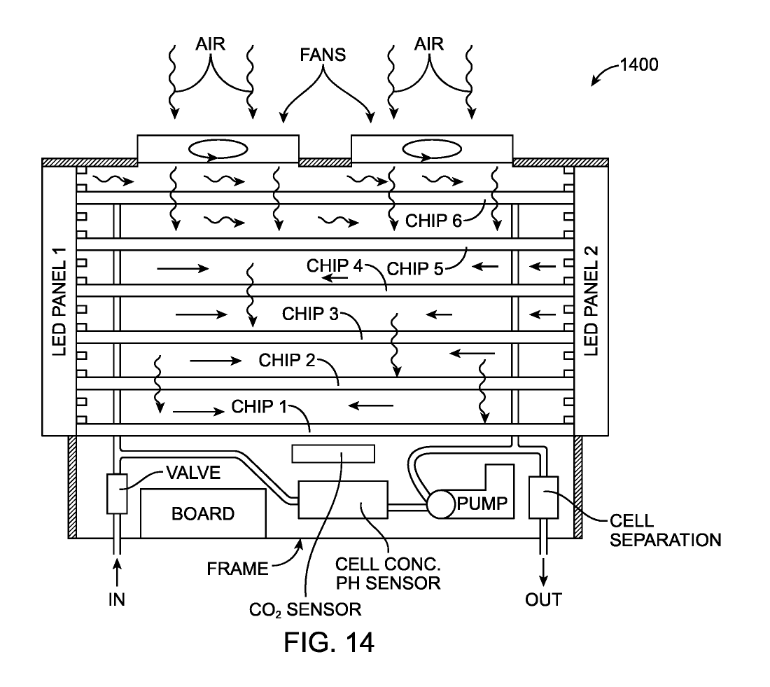

FIG. 14 provides another configuration of a multilayer microfluidic chip module, in accordance with embodiments. In this configuration comprising six microfluidic chips, the chips are aligned parallel to each other. LED panel 1 is located to one side of the microfluidic chips, and LED panel 2 is located to the other side of the microfluidic chips. Fans are located at the very top of the module, to circulate air though the module. At the bottom of the module is the chip base. It comprises a control board, a carbon dioxide sensor, a pH sensor and a cell concentration sensor. The chip base also comprises a pump to assist with fluid flow in and out of the microfluidic channels. Fluid flows into the module through an input and a valve into the chip base and through multiple tubings connected to the microfluidic chip modules. The tubings are also connected to the concentration and the pH sensors and the pump. A cell separator separates the fluids into live cells output and dead cells output as the fluids exit the chip base.

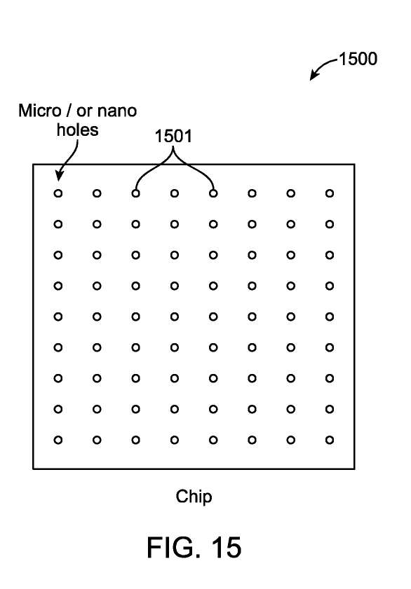

FIG. 15 provides an exemplary illustration of a microfluidic chip 1500 with micro-holes 1501, in accordance with embodiments. In some examples, the size of the holes can range from about 10 nm to about 100 nm. In some examples, the size of the holes can be at least about 10 nm. In some examples, the size of the holes can be at most about 100 nm. The size of the holes can be from about 10 nm to about 20 nm; from about 20 nm to about 30 nm; from about 30 nm to about 40 nm; from 40 nm to about 50 nm; from about 50 nm to about 60 nm; from about 60 nm to about 70 nm; from about 70 nm to about 80 nm; from about 80 nm to about 90 nm; from about 90 nm to about 100 nm. The use of micro-holes 1501 and/or nano-holes may improve the permeability and efficiency membranes. The holes 1501 may be in the membranes, which comprise silicon-based elastomers in some embodiments. The holes 1501 may result in higher gas permeability of the microfluidic chip. Higher permeability may increase the efficiency of the system.

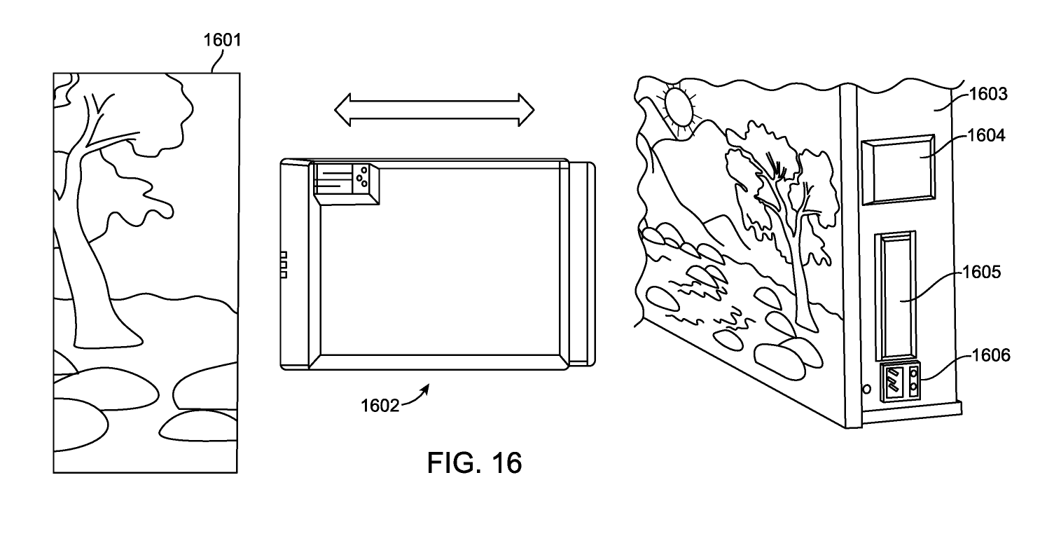

FIG. 16 provides an exemplary illustration of a system denoting the front design panel, the microfluidic panel behind the front design panel and a side view show the cartridge and the user interface, in accordance with some embodiments. In particular, FIG. 16 illustrates front design panel 1601. Behind design panel 1601 is a microfluidic system 1602. A side-view of the module shows the control system 1603, which comprises a control board 1604, cartridges 1605 and a user interface element 1606.

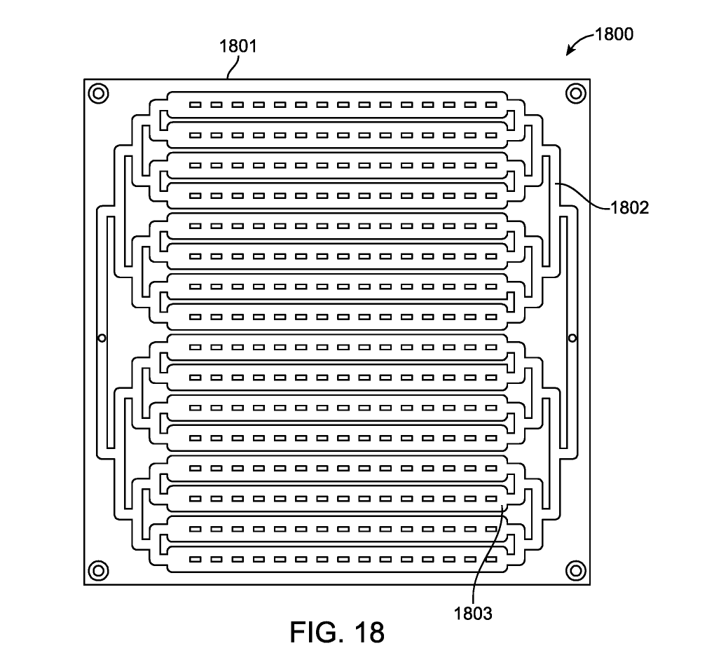

FIG. 17 provides an exemplary illustration of the arrangement of micro-channels in a microfluidic chip, in accordance with some embodiments. In particular, FIG. 17 provides an exemplary illustration of the arrangement of micro-channels in a microfluidic chip 1701, comprising of inputs and/or outputs 1702 which lead into multiple micro-channels 1703 configured to be parallel with each other. Additionally, FIG. 18 provides another exemplary illustration of the arrangement of micro-channels in a microfluidic chip, in accordance with some embodiments. In particular, FIG. 18 provides an arrangement of micro-channels in a microfluidic chip 1801, with inputs and/or outputs branching out 1802, and the branches feeding into multiple micro-channels 1803 arranged in parallel with each other.

Any improvement may be made in part or all of the systems, bioreactors, chips, chip modules and method steps. All references, including publications, patent applications, and patents, cited herein are hereby incorporated by reference. The use of any and all examples, or exemplary language (e.g., “such as”) provided herein, is intended to illuminate the disclosure and does not pose a limitation on the scope of the disclosure unless otherwise claimed. Any statement herein as to the nature or benefits of the disclosure or of the embodiments is not intended to be limiting, and the appended claims should not be deemed to be limited by such statements.

More generally, no language in the specification should be construed as indicating any non-claimed element as being essential to the practice of the disclosure. This disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contraindicated by context.

While some embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of removing a component from air, comprising,
(a) providing a microfluidic chip comprising a fluid flow path in fluid communication with at least one surface comprising at least one phototrophic organism;
(b) bringing air in contact with the at least one surface comprising said at least one phototrophic organism; and
(c) removing said component from said air with said at least one phototrophic organism.

2. The method of claim 1, wherein said bringing of (b) comprises subjecting said air to flow through said fluid flow path.

3. The method of claim 2, wherein said air is subjected to flow through said fluid flow path using a pump.

4. The method of claim 1, wherein said at least one phototrophic organism comprises a plurality of phototrophic organisms.

5. The method of claim 1, wherein said microfluidic chip comprises said at least one surface comprising said at least one phototrophic organism.

6. The method of claim 1, wherein said fluid flow path and at least one surface are included in at least one channel of said microfluidic chip.

7. The method of claim 1, wherein said component includes CO or CO₂.

8. The method of claim 1, wherein said recovering (c) comprises exposing said at least one phototrophic organism to light.

9. A microbial air purification system comprising an air exchange surface area of less than 1 m² and phototrophic organisms in contact with the air exchange surface area, wherein the phototrophic organisms lower a concentration of a component in air in a room having a volume greater than 1000 ft³ by at least 25% when the phototrophic organisms are exposed to light over a time period of at least 10 seconds.

10. The system of claim 9, wherein said phototrophic organisms are contained within microfluidic channels of a microfluidic chip.

11. The system of claim 9, wherein a light source is coupled to the microfluidic chip.

12. The system of claim 9, wherein the component is selected from the group consisting of a carbon oxide, a nitrous oxide, and a sulfur oxide.

13. A microbial air purification system, comprising:
a microfluidic chip having one or more microfluidic channels, wherein the one or more microfluidic channels contain phototrophic organisms cultured in a cell culture medium, wherein in response to light, the phototrophic organisms remove a component in air that is within the cell culture medium.

14. The system of claim 13, wherein the air is dissolved within the cell culture medium.

15. The system of claim 13, further comprising:
a light source that is operably connected to the microfluidic chip, wherein the light source is able to provide light to the microfluidic channels within the microfluidic chip.

16. The system of claim 13, further comprising:
a replaceable fluid cartridge that is attachable to the microfluidic chip, wherein the fluid cartridge has a fluid conduit that is operably connected to the microfluidic channels within the microfluidic chip.

17. The system of claim 16, wherein the fluid cartridge contains cell culture medium.

18. The system of claim 17, further comprising:
a pump that is capable of moving the cell culture medium from the fluid cartridge to the microfluidic chip.

19. The system of claim 13, wherein the air purification system is non-toxic.

20. A microbial purification system, comprising:
a microfluidic chip having one or more microfluidic channels, wherein the one or more microfluidic channels contain phototrophic organisms cultured in a cell culture medium, wherein the phototrophic organisms remove a gas contacting the phototrophic organisms in response to exposure to light.

21. The system of claim 20, further comprising:
a permeable membrane integrated within the microfluidic channels, wherein a first portion of the permeable membrane contacts the culture of phototrophic organisms, and wherein a second portion of the permeable membrane contacts air, wherein the permeable membrane provides an air exchange surface area.

22. The system of claim 20, further comprising:
a light source that is attached to the microfluidic chip, wherein the light source is configured to provide light to the microfluidic channels of the microfluidic chip.
